(19) United States
(12) Patent Application Publication
(10) Pub. No.: US 2025/0367658 A1
(43) Pub. Date: Dec. 4, 2025

(54) Systems And Methods For Semi-Quantitative And Quantitative Detection Of Metabolities Of Progesterone

(71) Applicant: Easy Healthcare Corporation, Darien, IL (US)

(72) Inventors: Xiaolian Liu, Darien, IL (US); Li Zou, Darien, IL (US)

(21) Appl. No.: 19/221,402

(22) Filed: May 28, 2025

Related U.S. Application Data
(60) Provisional application No. 63/652,529, filed on May 28, 2024.

(51) Int. Cl. B01L 3/00; G01N 33/74

(52) U.S. Cl. CPC B01L 3/5023; B01L 3/502715; G01N 33/743; B01L 2200/04; B01L 2200/148; B01L 2300/0654; B01L 2300/069

(57) ABSTRACT

A test system includes a test device and a calibration chart. The test includes a lateral flow assay that detects the presence of a target analyte. In response to exposure to the target analyte, the results area produces a visually identifiable test-line and a visually identifiable control-line, wherein the visually identifiable test-line increases in darkness according to an increased concentration level of the target analyte. The calibration chart includes a plurality of color blocks, wherein each of the color blocks is representative of a test-line color density that corresponds to a quantitative hormone concentration in the target substance. The test system further includes a user device that derives a quantitative result, tracks the quantitative levels over the time, and compares them to normal hormone trends and threshold values associated with defined risk levels for certain health conditions.

### BACKGROUND

The present subject matter relates generally to deriving reliable semi-quantitative or quantitative results from qualitative hormone and chemical analyte tests. Specifically, the present systems and methods provide qualitative hormone and chemical analyte test results using a test strip, cassette, or midstream product that is based on a non-competitive lateral flow assay (LFIA). The qualitative test result shows the visually identifiable test-line color density increases according to an increased concentration level of the target analyte. The semi-quantitative or quantitative results provided by the systems and methods taught herein can be derived from the qualitative hormone and chemical analyte test results by either: (1) manually comparing a test line color density from the qualitative test to an associated color chart; or (2) using a computing device running purpose-designed software to automatically interpret the results of qualitative hormone and chemical analyte tests. The semi-quantitative or quantitative hormone levels and chemical concentration levels derived from these systems and methods provide reliable quantitative measurements that can be used for subsequent analysis and recommendations.

Progesterone is an endogenous steroid reproductive hormone synthesized by the adrenal cortex and gonads, including the ovaries and testes. During a woman's normal menstrual cycle, the progesterone levels increase following ovulation, primarily produced by the corpus luteum during this phase. Therefore, the presence of progesterone or its metabolites, such as pregnanediol glucuronide (“PdG”), in the body can confirm the occurrence of ovulation after it has already taken place. This retrospective confirmation can be useful in many scenarios, such as tracking menstrual cycles, predicting fertility, and diagnosing infertility issues.

During the initial ten weeks of pregnancy, the ovarian corpus luteum secretes progesterone, which is subsequently replaced by the placenta during the later stages of pregnancy. Progesterone is crucial for sustaining the uterus during pregnancy, and its deficiency has been associated with preterm labor and miscarriage. The hormone's primary function during pregnancy is to lower vascular tone in the myometrium. Additionally, progesterone modulates the production of inflammatory mediators, including human T-cells, in the uterine cavity. Reduced levels of progesterone increase myometrial contractility and decrease immune response, thereby elevating the risk of miscarriage and premature delivery of the fetus. Further, progesterone is essential in the preparation of the uterus for pregnancy, with levels rising in response to the monthly release of an egg during a typical menstrual cycle. Specifically, progesterone stimulates the thickening of the uterine lining, creating an optimal environment for the implantation and growth of a fertilized egg. This process enables successful conception and pregnancy to occur.

Progesterone levels typically begin to rise 24-36 hours after ovulation. In the event of non-pregnancy, progesterone levels in the body decline around five to ten days after ovulation, resulting in the thinning of the uterine lining. This process causes the shedding of excess blood and tissue, marking the onset of the menstrual period. FIG. 1 illustrates the typical fluctuation of progesterone levels throughout a female's normal menstrual cycle. The information provided in FIG. 1 is based on data provided at https://thriva.co/hub/womens-health/ovulation-blood-test-progesterone (April 2024).

In the event of pregnancy, progesterone levels increase to sustain the gestation process, reaching levels approximately ten times higher than normal. Elevated levels of progesterone serve to prevent the uterus from contracting, thereby avoiding the risk of pre-term labor. Much of the progesterone required to support a healthy pregnancy is produced by the placenta, an organ that grows in the uterus to facilitate the provision of nutrients and oxygen to the developing fetus. FIG. 2 illustrates the typical progesterone level changes during a woman's gestational period. The information provided in FIG. 2 is based on data disclosed in Plasma estrone, estradiol, estriol, progesterone, and 17-hydroxyprogesterone in human pregnancy. I. Normal pregnancy., 112 (8) Am. J. Obstet. Gynecol. 1095-1100 (April 1972).

Numerous variables can influence progesterone levels and cause fluctuations in the average value of the levels. Factors that impact the accuracy of progesterone level tests may include, for example: the timing of the menstrual cycle; presence or absence of ovulation; the specific laboratory conducting the test; the timing of the blood sampling relative to meals; and whether the sample is collected during the morning or afternoon. Traditionally, serum progesterone levels are measured during the mid-luteal phase, which is about seven days after ovulation. The recommended level of serum progesterone for optimal fertility is equal to or greater than 10 ng/mL. However, one major challenge in progesterone monitoring is the pulsatile release of the hormone from the corpus luteum, which leads to fluctuations in serum levels ranging from 2 ng/ml to 40 ng/ml in a 24-hour period in the same healthy individual. Due to its frequent fluctuations, a limited number of isolated data points may not be sufficient for meaningful analysis. Accordingly, to extract valuable information from the data, it is helpful to analyze a woman's progesterone level trend over a period of time.

In the female body, progesterone levels vary depending on the stage of the menstrual cycle or pregnancy. During the early menstrual cycle, serum progesterone levels are typically 1 ng/ml or lower. Before ovulation, progesterone levels usually remain below 1 ng/ml. During the mid-cycle, about 7-10 days after ovulation, progesterone levels can rise above 8-10 ng/ml, even up to 25 ng/ml or higher. During the first, second, and third trimesters of pregnancy, progesterone levels range from 11.2 to 90 ng/ml, 25.6 to 89.4 ng/ml, and 48.0 to 150 to 300 or more ng/ml, respectively. Although progesterone levels tend to be higher during pregnancy, in non-pregnant patients, the levels can reach up to 25 ng/ml. During pregnancy, progesterone levels should be maintained at least above 10 to 12 ng/ml to increase the chances of a successful pregnancy outcome.

PdG is the primary metabolite of progesterone present in urine. Recent research has demonstrated the utility of PdG as a non-invasive marker for monitoring mid-luteal activity. By measuring PdG levels in urine, clinicians can relatively accurately monitor the activity of progesterone in the body without the need for invasive blood tests.

In the field of substance testing or reproductive hormone testing, such as PdG testing, various types of assays are available including, but not limited to, competitive assays, sandwich assays, reverse sandwich assays, multiplex assays, and nucleic acid amplification-based assays. These assays can be divided into categories based on how the test results are presented. The first category is a qualitative test that indicates a positive result when only the control line (i.e., the C-line), and not the test line (i.e., the T-line), is visually present. The positive result is only capable of indicating whether the testing hormone level is above a predefined cutoff level. Competitive assays, such as some existing PdG test and estrone-3-glucuronide (“E3G”) tests are in the first category. In the second category, the T-line appears when the result is positive (i.e., when the hormone level is above the cutoff level) or the substance presents. The intensity of the T-line is relatively proportional to the amount of target analyte in the urine sample. This second category can be a semi-quantitative or quantitative test, and existing tests include luteinizing hormone (“LH”) or hCG hormone tests. Noncompetitive assays such as sandwich assays, reverse-sandwich assays, or nucleic-acid amplification-based assays are included in this second category.

Laboratory-based testing, such as the enzyme-linked immunosorbent assay (ELISA), is an accurate method for testing progesterone levels. The ELISA method involves detecting and quantifying the presence of specific progesterone antibodies in a serum, urine, or other sample. Accordingly, ELISA tests require lab equipment, careful handling, and precise techniques to ensure accurate results, so it could be inconvenient, time-consuming, and expensive compared to qualitative or semi-quantitative LFIA tests. Additionally, progesterone levels can fluctuate throughout the day, so the results from a single test may not provide an accurate representation of a person's average progesterone levels on a given day.

Similarly, some existing home tests can be inconvenient and/or expensive. For example, home tests, such as devices using fluorescent-based testing, typically require a special device for reading the test results. For example, some known home tests require an additional device to interpret the results of a fluorescent-based PdG test.

There are known qualitative tests for detecting PdG presence; however, these tests use a competitive lateral flow immunoassay (competitive LFIA) and can only determine whether the level of PdG in a sample is above or below a certain threshold, resulting in a binary positive or negative result. Specifically, when the PdG rises beyond the cutoff level, the T-line disappears, and it is impossible for the user to determine how high the PdG level is. For example, some tests employ PdG testing strips that become positive at five μg/mL, which is equivalent to approximately 10 ng/ml of progesterone in blood, to indicate whether ovulation has happened. FIG. 3 illustrates an exemplary prior art test strip in which the test strip has a control, or C-line, and a test, or T-line. When the PdG level is lower than the cutoff level and the result is negative, both the C-line and the T-line present. When the PdG level is the same as or above the cutoff level and the result is positive, the C-Line presents and the T-line does not.

Based on recent studies, it is believed that PdG urine tests can be used as a reliable and non-invasive method, as an alternative to serum testing, for detecting progesterone levels in a variety of scenarios. Such scenarios include monitoring fertility, diagnosing ovulation disorders, monitoring early pregnancy, and assessing the effectiveness of progesterone therapy.

Accordingly, there is a need for a convenient, inexpensive, quantitative, non-invasive PdG test to accurately measure the quantitative PdG level or progesterone level. There is also a need to provide users with greater flexibility and convenience to take the test multiple times a day to monitor their PdG levels at relatively low cost. Further, there is a need to provide women the ability to track their quantitative PdG level trends and changes, which may enable additional medical benefits, such as: more convenient monitoring of the health of early pregnancy; detecting the risk of miscarriage and ectopic pregnancy; and assisting in fertility treatment. There is a need for such tests to be used in conjunction with a smart device and/or software, to assist users in automatically reading, interpreting, and managing their PdG level data more conveniently. There is a further need for electronic devices and/or software to provide preliminary analysis and to alert the individual of potential health issues. Furthermore, there is a need for individuals to be able to conveniently share this data with physicians, enabling diagnostic consultations.

### BRIEF SUMMARY OF THE INVENTION

The present disclosure provides a test system that derives reliable semi-quantitative or quantitative results from qualitative hormone and chemical analyte tests. Specifically, the present systems and methods provide qualitative hormone and chemical analyte test results using, for example, a test strip or cassette product that is based on a non-competitive lateral flow assay (LFIA). The qualitative test result shows the visually identifiable test-line color density increases according to an increased concentration level of the target analyte. The semi-quantitative or quantitative results provided by the systems and methods taught herein can be derived from the qualitative hormone and chemical analyte test results by either: (1) manually comparing a test line color density from the qualitative test to an associated color chart; or (2) using a computing device running purpose-designed software to automatically interpret the results of qualitative hormone and chemical analyte tests. The semi-quantitative or quantitative hormone levels and chemical concentration levels derived from these systems and methods provide reliable quantitative measurements that can be used for subsequent analysis and recommendations.

In a primary embodiment, the test system includes a testing strip, cassette, or midstream stick with a non-competitive lateral flow assay (LFIA) test area. The test area provides semi-quantitative test results, which can be interpreted into numerical values manually by comparing a color density of a test line to a color chart. Alternatively, a processing device running appropriate software, such as a smartphone running an associated mobile app, may be configured to automatically interpret the semi-quantitative results into quantitative hormone levels or chemical concentration levels. When using software to interpret the test results, a further system, such as the quantitative hormone and chemical analyze test result systems and methods disclosed in U.S. Pat. No. 11,519,909, the entirety of which is hereby incorporated by reference, can be used to identify the test area, comparing the T-line and C-line color densities to calculate a T/C ratio, then comparing the T/C ratio with a data structure that maps calibrated T/C ratios to quantitative hormone levels, which is created based on the color chart and embedded in the software, to finally determine the quantitative hormone levels. More specifically, with respect to calculating the T/C ratio, the software may compare the greyscales of the T-line and the C-line to the white background of the test result image to control for ambient interference and lighting conditions to more accurately determine respective darkness values. The software may then calculate a color intensity ratio as a ratio of the darkness value of the T-line to the darkness value of the C-line. This interpretation process taught in U.S. Pat. No. 11,519,909 does not need additional hardware devices or accessories, i.e., lighting control accessories, regardless of ambient interference and lighting conditions.

In one embodiment of the systems and methods taught herein, the test system comprises a test body including a test area, a results area, and a control area, with the test area including a lateral flow assay that detects the presence of a target analyte. The test system also includes a calibration chart including a plurality of color blocks. In response to exposure to the target analyte within a target substance, such as urine, the results area produces a visually identifiable test-line (T-line) having a test-line color density and a visually identifiable control-line (C-line) having a control-line density. The visually identifiable test-line increases in darkness according to an increased concentration level of the target analyte. Each of the color blocks of the calibration chart is representative of a test-line color density that corresponds to a quantitative hormone concentration or level in the target substance.

In some embodiments, the test area is a non-competitive lateral flow assay for determining the concentration of the target analyte, and the target analyte is a progesterone metabolite. In further embodiments, the target analyte could be a pregnanediol such as PdG, pregnanolone, and pregnanetriol. The target analyte may be present in a sample of urine or serum.

In some embodiments, the test body further includes a line separating the test area and the results area, wherein the line is indicative of a maximum submerged level.

The test system may further include: a user device including a camera, a processor, a display, and memory storing program instructions, wherein, in response to executing the program instructions, the processor: receives an image of the test area from the camera, the image including the visually identifiable test-line and the visually identifiable control-line; determines a test-line value defined as a numerical value of a color density of the test-line in the image; determines a control-line value defined as a numerical value of a color density of the control-line in the image; and calculates a quantitative hormone concentration in the target substance using the numerical value of the color density of the test-line in the image and the numerical value of the color density of the control-line in the image.

Calculating a quantitative hormone concentration in the target substance using the numerical value of the color density of the test-line in the image and the numerical value of the color density of the control-line in the image may include: calculating a T/C ratio defined as a relative value of the test-line value to the control-line value; and determining a quantitative hormone level based on the comparison between the T/C ratio and a data structure that maps calibrated T/C ratios to quantitative hormone levels.

In response to executing the program instructions, the processor may further present visualized data through the display indicative of one or more T/C ratio trends or one or more quantitative hormone level trends.

The test system may further comprise a user device including a processor, a display, and memory storing program instructions, wherein, in response to executing the program instructions, the processor: receives quantitative hormone levels at one or more specific stages of a menstrual cycle or a pregnancy derived from the test device and calibration chart; and compares the quantitative hormone levels at one or more specific stages of a menstrual cycle or a pregnancy to one or more threshold values associated with risk levels of certain health conditions.

The one or more threshold values associated with risk levels of one or more health conditions may be determined based on scientific research and data analysis in combination with a user's personalized data derived from the test device and calibration chart.

The display may provide an indication of a potential health condition risk derived from a comparison between the quantitative hormone levels at one or more specific stages of a menstrual cycle or a pregnancy derived from the test device and calibration chart and one or more screening data sets.

In another embodiment, a test system includes: a test device including a test area, a results area, and a control area, wherein the test area of the body includes a lateral flow assay that detects the presence of a target analyte in a target substance, wherein, in response to exposure to the target analyte, the results area produces a visually identifiable test-line having a test-line color density and a visually identifiable control-line having a control-line density, wherein the visually identifiable test-line increases in darkness according to an increased concentration level of the target analyte; and a user device including a camera, a processor, a display, and memory storing program instructions, wherein, in response to executing the program instructions, the processor: receives an image of the test area from the camera, the image including the visually identifiable test-line and the visually identifiable control-line; determines a test-line value defined as a numerical value of the test-line color density; determines a control-line value defined as a numerical value of the control-line color density; calculates a T/C ratio defined as a relative value of the test-line value to the control-line value; determines a quantitative hormone level based on the comparison between the T/C ratio and a data structure that maps calibrated T/C ratios to quantitative hormone levels, and presents visualized data through the display indicative of one or more T/C ratio trends or one or more quantitative hormone level trends.

The T/C ratios or the quantitative hormone levels at one or more specific stages of a menstrual cycle or a pregnancy may be compared to one or more threshold values associated with risk levels of certain health conditions and presented in the visualized data. The one or more threshold values associated with risk levels of one or more health conditions may be determined based on scientific research and data analysis in combination with a user's personalized data. The one or more health conditions may include, for example, one or more of: a miscarriage, an ectopic pregnancy, an ovarian tumor, or an adrenal gland problem.

The visualized data presented through the display may further include a comparison between the one or more T/C ratio trends or one or more quantitative hormone level trends and a normal hormone level trend. The visualized data presented through the display may further include an indication of a potential health condition risk derived from a comparison between the one or more T/C ratio trends or one or more quantitative hormone level trends and one or more screening data sets.

Overall, the semi-quantitative or quantitative PdG test taught herein offers a cost-effective and user-friendly method to generate more regular, routine, and/or continuous progesterone level data, presenting numerous applications for personal reproductive health management, for medical condition screening or risk assessment, and for aiding diagnostics in medical or clinical contexts. With respect to personal reproductive health management, the subject matter taught herein can provide tools to track and confirm ovulation, monitor menstrual cycles, track the health of early pregnancy, calculate fertility windows, and enable other reproductive hormone tracking purposes. Further, tracking personalized hormone level analysis against aggregated test results can aid in various health or medical condition screening or risk assessment or aiding of diagnostics, including, but not limited to, assessing the risk of miscarriage; assessing the risk of or screening or even aiding the diagnostics of an ectopic pregnancy; monitoring high-risk pregnancies; screening or assessing the risk of or aiding the diagnostics of the ovarian cancer or adrenal gland disorders, as high levels of progesterone may be indicative of adrenal gland dysfunction in both males and females; aiding to determine the optimal egg retrieval times and facilitating embryonic implantation in nature IVD process; screening or aiding to identify the cause of female infertility or subfertility or cause of female reproductive hormone disorder; and determining whether fertility treatments are effective.

One advantage of the present systems and methods is providing more reliable and accurate hormone and/or analyte concentration level results.

A further advantage of the present systems and methods is enabling the use of a variety of conventional hormone level tests (including, but not limited to, strip tests and cassette ovulation tests) to produce quantitative results.

Another advantage of the present disclosure is that the test strip or cassette will produce both a T-Line and a C-line when the concentration level of PdG is above a certain cutoff level, and the color density or intensity of the line becomes darker when the concentration level of PdG level in a urine sample is higher.

Another advantage of the present disclosure is that the test does not require a separate device to read the results, so users can obtain the quantitative chemical concentration levels at point-of-collection without buying additional expensive devices or accessory attached to the phone or taking expensive and inconvenient lab tests. Accordingly, the present invention provides easy and low-cost methods to obtain a large number of accurate, quantitative progesterone concentration level data points in a cycle, including enabling user to take a test multiple times per day. Obtaining large amounts of continuous data points can aid in avoiding and minimizing progesterone fluctuation issues by skipping the abnormal data points in a day and making it possible to produce more continuous progesterone level charting.

Another advantage of the present disclosure is to provide monitoring of the progesterone level during early stages of pregnancy or for early pregnancy detection. For example, monitoring the quantitative progesterone level can help monitor or screen for pregnancy risks such as ectopic pregnancy or miscarriage. Specifically, the present invention can track the trend of PdG levels relevant to the number of days of pregnancy. If the patient's estimated progesterone level based on the PdG level remains at a lower than normal/recommended level when it should be at a higher level based on the pregnancy stage, this may indicate a potential health risk and prompt the user to seek medical attention. Furthermore, by monitoring progesterone levels and trends, the present invention may enable a user to seek support for progesterone supplementation when the user notices that they have inadequate progesterone secretion. Inadequate progesterone secretion during early pregnancy has been identified as a potential cause of recurrent miscarriages, and the present invention may be used to provide evidence that the administration of progesterone can decrease the rate of subsequent miscarriages in individuals with unexplained recurrent miscarriages, when compared to historical data.

A further advantage of the present disclosure is it can be used with smart device and/or software (though not mandatory) to read, interpret, and analyze PdG level test results, thereby improving convenience and minimizing human error. In some embodiments, the software can translate the color densities of the test lines into the concentration level of the target analyte, using the T/C ratio method described above with respect to U.S. Pat. No. 11,519,909, without additional hardware devices or accessories, regardless of ambient interference and lighting conditions. The smart device and/or software can generate graph and/or chart based on the test results obtained through the present disclosure, the concentration level of the target analyte, along with other hormone levels measured by diverse methods and input by users. This capability allows the present disclosure to be applied in various contexts, as described herein.

Additional objects, advantages, and novel features of the examples will be set forth in the description which follows, and in part will become apparent to those skilled in the art upon examination of the following description and accompanying drawings or may be learned by production or operation of the examples. The objects and advantages of the concepts may be realized and attained by means of the methodologies, instrumentalities, and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a graph demonstrating a fluctuation of progesterone levels over a typical menstrual cycle.

FIG. 2 is a graph demonstrating fluctuation of progesterone levels over a typical pregnancy.

FIG. 3 is an illustration of various results provided by prior art qualitative PdG test strip using competitive lateral flow immunoassay.

FIG. 4 illustrates an example of a test strip and color chart of a test system taught by the present disclosure,

FIG. 5 illustrates an example procedure for performing a test using the test system of FIG. 4.

FIG. 6 illustrates the results of different progesterone (or PdG) test strips of the test systems of FIG. 4.

FIG. 7 illustrates an example calibration color chart containing color blocks that correspond to different progesterone concentration levels of the test system of FIG. 4.

FIG. 8 illustrates an example of a normal pregnancy progesterone progression chart displayed through a mobile application running on a smartphone.

FIG. 9 illustrates an example of a pregnancy progesterone progression chart showing a normal pregnancy in comparison to a pregnancy with ectopic pregnancy risk displayed through a mobile application running on a smartphone.

FIG. 10 illustrates an example of a progesterone progression chart showing a normal ovulation in comparison to an ovulation of a woman having high risk of an ovarian tumor or problems with the adrenal glands displayed through a mobile application running on a smartphone.

FIG. 11 illustrates an example of a pregnancy progesterone progression chart showing a normal pregnancy in comparison to a pregnancy with a high risk of miscarriage displayed through a mobile application running on a smartphone.

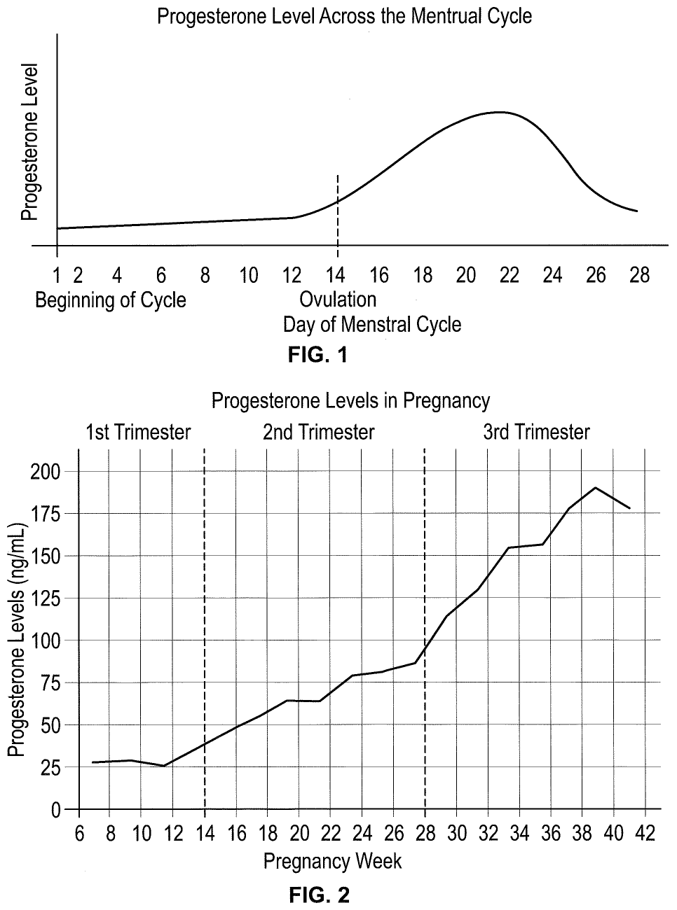

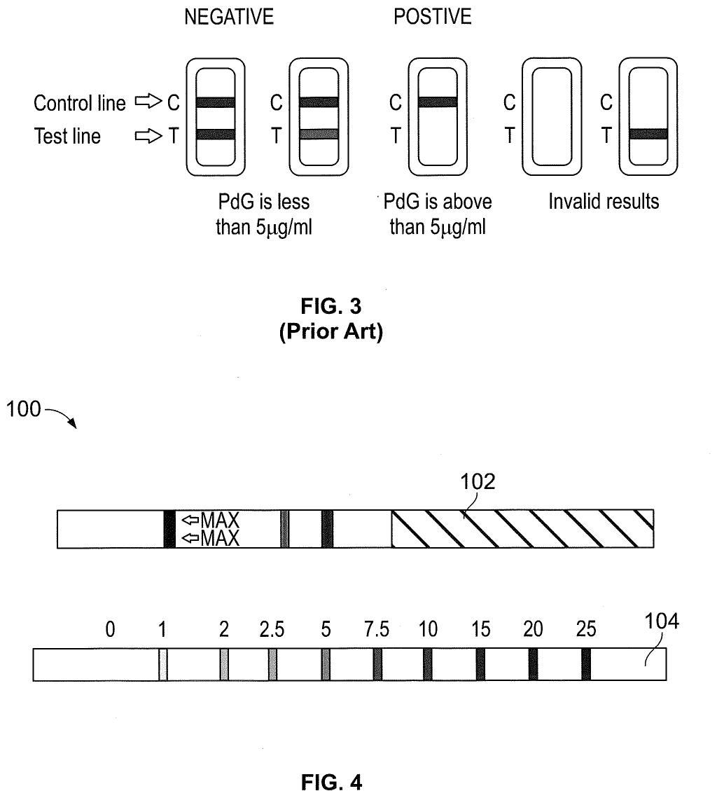

### DETAILED DESCRIPTION OF THE INVENTION

As described in further detail herein, the subject matter presented herein provides a test system that derives reliable semi-quantitative or quantitative results from qualitative hormone and chemical analyte tests. Specifically, the present systems and methods provide qualitative hormone and chemical analyte test results using, for example, a test strip or cassette product that is based on a non-competitive lateral flow assay (LFIA). The qualitative test result shows the visually identifiable test-line color density increases according to an increased concentration level of the target analyte. The semi-quantitative or quantitative results provided by the systems and methods taught herein can be derived from the qualitative hormone and chemical analyte test results by either: (1) manually comparing a test line color density from the qualitative test to an associated color chart; or (2) using a computing device running purpose-designed software to automatically interpret the results of qualitative hormone and chemical analyte tests. The semi-quantitative or quantitative hormone levels and chemical concentration levels derived from these systems and methods provide reliable quantitative measurements that can be used for subsequent analysis and recommendations.

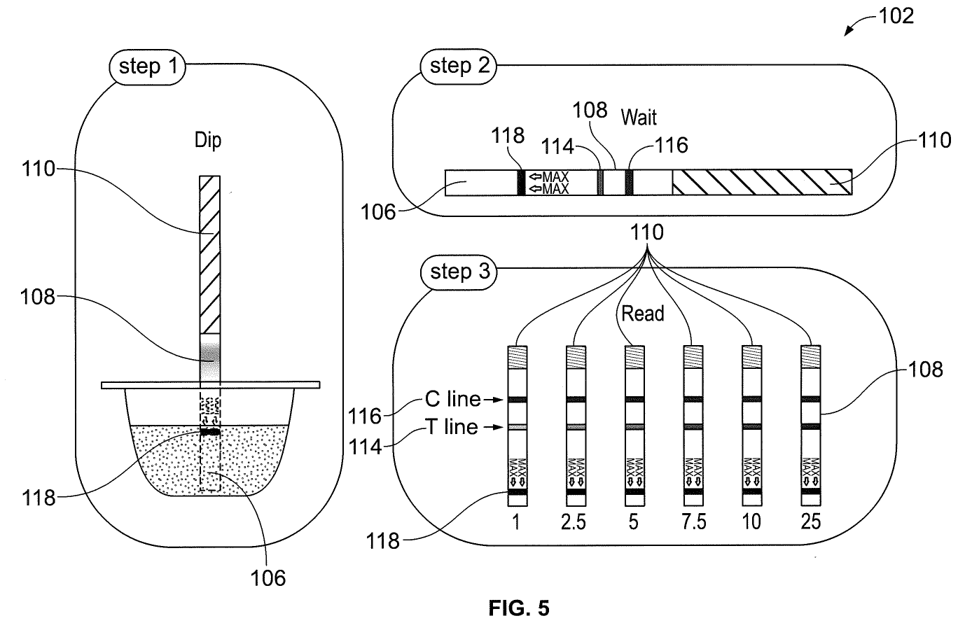

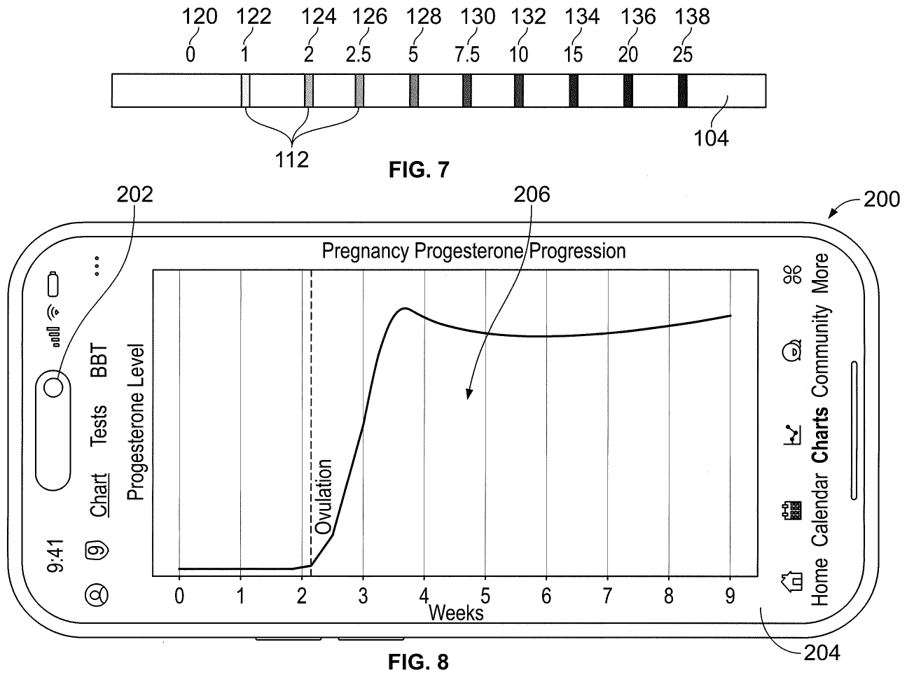

Referring to FIGS. 4-7, an exemplary test system 100 includes a test device 102 (e.g., test strip 102) and a calibration chart 104. Referring to FIG. 5, the test device 102 includes a test area 106, a results area 108, and a test handling area 110, and, as shown in FIG. 7, the calibration chart 104 (e.g., calibration color chart 104) includes a plurality of color blocks 112. The test area 106 of the test device 102 includes a lateral flow assay that detects the presence of a target analyte. In response to exposure to the target analyte, the results area 108 produces a visually identifiable test-line 114 having a test-line color density and a visually identifiable control-line 116 having a control-line density. Each of the color blocks 112 is representative of a test-line color density that corresponds to a quantitative hormone concentration in the target substance.

In some embodiments, the test area 106 further comprises a maximum (“max”) line 118 located between the test area 106 and the results area 108 to indicate to the user the end point of the test area 106. During use, the max line 118 indicates that the user should not dip the test area 106 beyond the max line 118, as shown in FIG. 5.

As shown in the example procedure shown in FIG. 5, in the first step, a user dips an unused test device 102 into a sample of urine. In particular, the user holds the handle portion of the test device 102 and dips the test area 106 of the unused test device 102 into the urine sample. Prior to submerging the test area 106 of an unused test device 102, the results area 108 does not visibly identify a T-line 114 or a C-line 116.

In this example, the test area 106 comprises a non-competitive lateral flow assay (LFIA) to react in response to the presence of a target analyte (e.g., PdG). In one example, the LFIA is a sandwich or reverse sandwich assay format or other non-competitive LFIA assay type. In a sandwich assay, two different antibodies can be used to bind to the target analyte. In the reverse sandwich assay, the analyte could be used to capture a single antibody. The non-competitive LFIA may be configured to detect one or more of the progesterone metabolites in the urine, including a pregnanediol such as PdG, pregnanolone, and pregnanetriol.

In a second step, the user removes the test area 106 from the urine sample and waits a short period of reaction time such as, for example, 120 seconds for results to appear on the results area 108. The results are visually identifiable by changes in the C-line 18 and the T-line 20.

In a third step, the results of a valid test are visually identifiable on the results area 108 of the test device 102. For example, a valid test will visually present a solid and stable C-line 116 and may or may not present a T-line 114. In the present embodiment, a test device 102 that does not present a solid C-line 116 is an invalid test. The test device 102 will not visually present a T-line 114 when there is no, or almost no, target analyte present in the urine sample. The test device 102 visually presents a T-line 114 when a target analyte (e.g., PdG) is present in the urine sample. The T-line 114 will vary in color density depending on the concentration level of the target analyte, as shown in FIG. 5, step 3, with a higher concentration level of target analyte resulting in a darker color density.

The present systems and methods are valuable and useful in that they produce a semi-quantitative or quantitative hormone and/or chemical concentration levels that may be used to track the progression and trend of hormone changes (e.g., ovulation hormones, including PdG) over time. With the present systems and methods, a user can test multiple times a day without the use of expensive or time-consuming extraneous equipment or devices. The LFIA-based test system 100 presented herein may be used to provide a semi-quantitative test that allows for useful estimation of the concentration levels of a target analyte present in a urine sample. The estimate is achieved by comparing the color density of the T-line 114 of the test result based on a physical or electronic calibration curve or mapping color chart (e.g., the calibration chart 104), which is generated based on analysis of testing samples performed on various standard progesterone concentrations. As shown, the calibration curve or mapping color chart converts the qualitative result of color intensity (e.g., color, darkness, lightness, etc.) of the T-line 114 and the C-line 116 into a quantitative chemical concentration level based on comparing the color density of the T-line 114 to the standardized calibration chart 104 shown in FIG. 7.

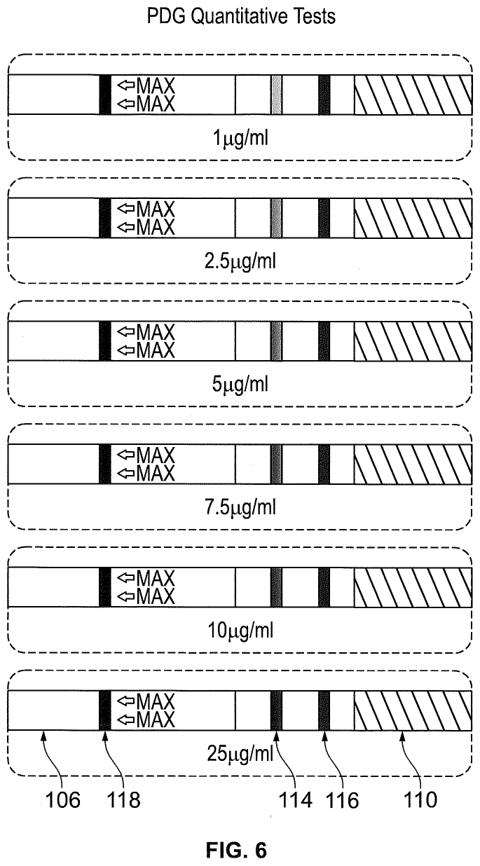

Regardless of the results of a valid test (i.e., test device 102 indicates that a target analyte is present or is not present in the urine), the C-line 116 is a solid and relatively stable line. However, as shown in FIG. 6, higher chemical concentration levels correspond to darker color densities of the T-line 114. Specifically, the T-line 114 presents as one color, but the possible colors that the T-line 114 can vary on a gradient. For example, as shown in FIG. 5, the T-line 114 color density corresponds to one of six chemical concentration levels. In this example, the test device 102 can visually present chemical concentrations in increments up to 25 μg/mL.

If the test area 106 does not detect the presence of the target analyte in the urine sample, a T-line 114 will not form, and the lack of a T-line 114 indicates, as shown in FIG. 7, that there is 0 μg/ml, close to 0 μg/mL 120, or trace amounts of target analyte chemical concentration. Contrastingly, the test device 102 will present a T-line 114 when the test area 106 detects the presence of a target analyte. In those circumstances, as shown in FIG. 6, the color density of the T-line 114 will correspond to a chemical concentration. In the example shown in FIG. 6, there are visually distinguishable color densities shown for detection of 1 μg/mL 122, 2 μg/mL 124, 2.5 μg/mL 126, 5 μg/mL 128, 7.5 μg/mL 130, 10 μg/mL 132, 15 μg/mL 134, 20 μg/ml 136, or 25 μg/mL 138. For example, if the testing area 14 detects a target analyte concentration of approximately 1 μg/mL 26, the T-line 20 will form a very light color density, but if the test area 106 detects a target analyte concentration of approximately 25 μg/mL 138, then the color density of the T-line 114 will have the darkest color density.

As further shown in FIG. 7, to determine the estimated quantitative hormone levels (e.g., PdG concentration levels) present in the user's urine sample, the user compares the color density of the T-line 114 relative to a standard calibration chart 104. Specifically, the color density of the T-line 114 corresponds to one of the color blocks on the calibration chart 104 provided with the test system 100. Each color block on the calibration chart 104 corresponds to a different progesterone concentration level. In this embodiment, the color density of the T-line 114 on the test device 102 is relatively proportional to the concentration level of the target analyte, which is one of the progesterone metabolites in the urine such as pregnanediol, prenanolone, or pregnanetriol. If the test area 106 does not detect the presence of the target analyte in the urine sample, a T-line 114 will not form, and the lack of a T-line 114 indicates that there is a 0 μg/mL 120 or nearly 0 μg/mL 120 target analyte chemical concentration.

In some embodiments, the systems and methods taught herein provide semi-quantitative results by providing the user with estimated quantitative hormone levels or ranges based on the user's manual comparison of the test line color density with the calibration chart 104. In other embodiments, the user can replace, or supplement, a manual comparison to the calibration chart 104 by using a digital image of the results area 108 and software to automatically read and interpret the semi-quantitative results into quantitative hormone levels or chemical concentration level.

For example, using a device including a camera, a processor, a display, and memory storing program instructions that, when run on the processor, cause the device to analyze a color density ratio between a T-line 114 and a C-line 116 in conjunction with a test device 102, the quantitative PdG level may be automatically determined by the device without any need to manually compare the T-line 114 to the calibration chart 104. In one embodiment, the device reads the color density of the T-line 114 in an image captured by the camera and automatically associates the color density of the T-line 114 with a concentration level of the target analyte based on calibration curving or mapping using the T/C ratio method described, for example, in U.S. Pat. No. 11,519,909. Implementing these systems and methods requires no additional hardware device or accessory to control the lighting or background to interpret the test results, regardless of ambient interference and lighting conditions. This type of automatic interpretation of the test area 108 may also improve the efficiency and accuracy of determining quantitative results. By incorporating this type of software-based analytical solution into the systems and methods described herein, the semi-quantitative, LFIA-based test device 102 of the present disclosure becomes a quantitative test providing accurate PdG concentration levels directly from the test device 102 results.

In some embodiments, the systems and methods taught herein leverage software-based analysis of test results from a test device 102 to perform deeper analysis and draw further conclusions. For example, the systems and methods taught herein may provide a device including a camera, a processor, a display, and memory storing program instructions that, when run on the processor, cause the device to process the test results with reference to one or more data models and along with additional data (provided by the user, provided by a third-party, or derived from statistical models) related to one or more other hormones levels to evaluate and assess trends of hormones levels changes. Such analysis may offer a preliminary assessment of various health-related aspects. For example, such analysis may help determine whether the user has ovulated in a specific month or may help in assessing the risk of certain health conditions, such as Polycystic ovary syndrome (PCOS). The analysis performed by the systems and methods can then be used to generate visual representations of the data, such as graphs and charts, to display the test results and analysis to both users and physicians and further improving the interpretability of the data.

For example, FIG. 8 illustrates mobile device 200 that includes a camera 202 (a rear-facing camera is also located on the backside of the device), a processor (inside the mobile device 200), a display 204, and memory (inside the mobile device 200) storing program instructions that may be run to perform tasks related to the systems and methods described herein. As further shown in FIG. 8, the display 202 illustrates a chart representative of PdG quantitative levels recorded over the weeks of a normal cycle into the first few weeks of early pregnancy 206. The data for the chart 206 shown in FIG. 8 may be specific to the user having been produced by the test system 100 or derived from other personal health records. Alternatively, the data for the chart 206 shown in FIG. 8 may be derived from a more generalized (i.e., not personal) data model, such as one representing a general population. For example, the chart 206 shown in FIG. 8 may be derived from aggregated and anonymized data collected from other users of the systems and methods. In some examples, the data for the chart 206 shown in FIG. 8 may be derived from a more generalized data model and then filtered using various demographic filters (e.g., age, health conditions, lifestyle choices, medications, etc.).

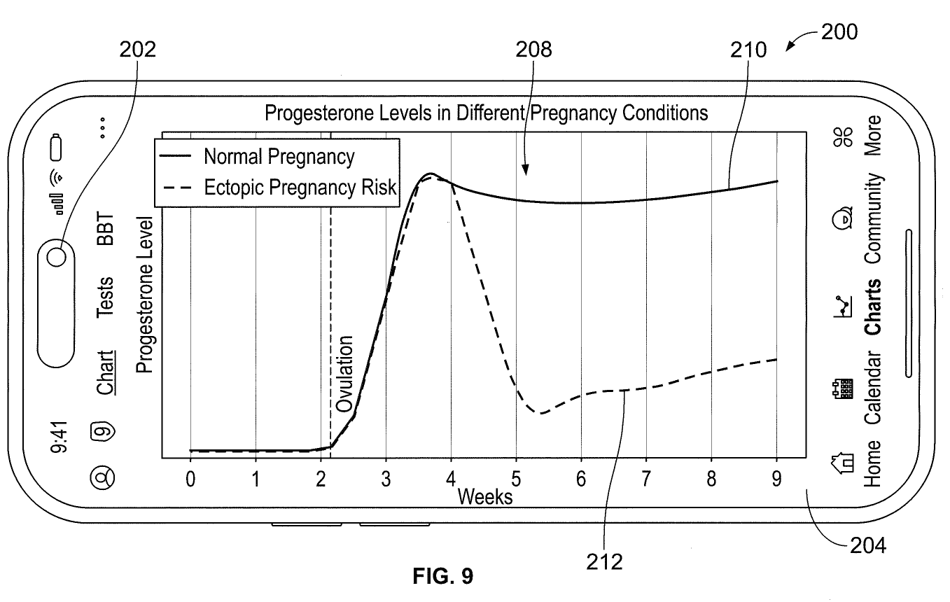

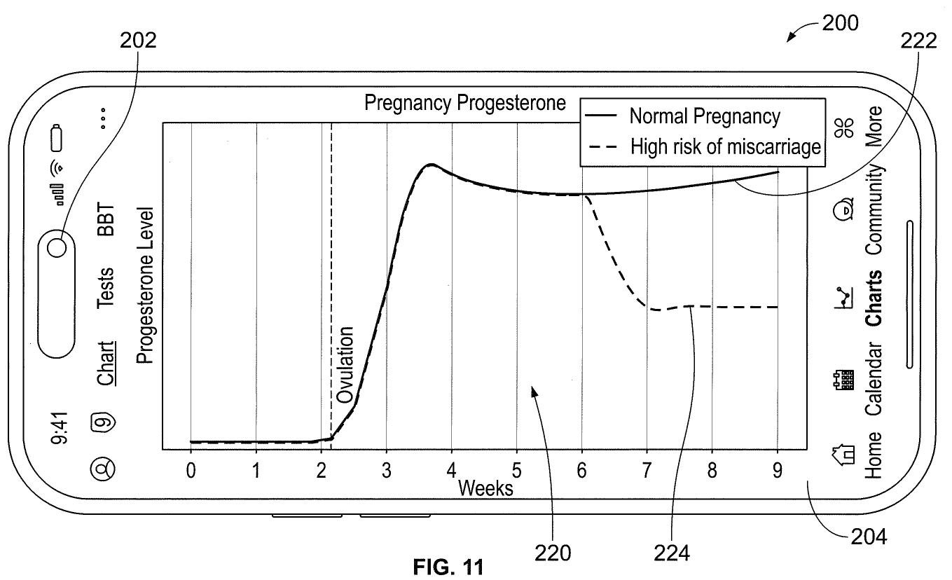

Whether the chart representative of PdG quantitative levels recorded over the weeks of a normal cycle into the first few weeks of early pregnancy 206 shown in FIG. 8 was derived from the personal data of the user, from data representative of the general population, from anonymized and aggregated data from other users of the systems and methods, or otherwise, the chart 206 may be used as a basis for comparison against newly plotted user data, as shown, for example, in FIGS. 9 and 11. As such, the systems and methods provided herein may help provide individual users and medical professionals tools useful for evaluating data, health screening, risk assessment, aiding in diagnosis, trend analysis, and prediction, as described further herein.

Specifically, with respect to analysis of serum progesterone quantitative levels, medical professionals have found these quantitative levels are valuable in many health and medical condition screenings or diagnoses. However, because a typical serum progesterone test may not be convenient and/or may be expensive, it has not been easy to use progesterone levels for screen or early detection of some health and medical conditions. The systems and methods taught in this disclosure enable users to more efficiently obtain quantitative progesterone levels based on a low-cost urine test, enabling users and healthcare professionals to collect a far greater number of data points representative of the quantitative progesterone level and to chart and monitor the user's hormone trends in comparison to the normal trends in a cycle or in comparison to various pregnancy stages (whether against personal historical data or more generalized data from other users). The software and mobile application allow for the analysis, personal screening, and/or assessment of the risk of some health conditions and can prompt messages to be displayed on a user interface indicative of the potential risk and the risk level comparing to a group of population with similar profile.

For example, the test system 100 of the present application may be used to screen or assess the risk of an ectopic pregnancy. According to some research studies, the average progesterone levels for women experiencing an ectopic pregnancy are significantly lower than the average progesterone levels for women experiencing normal pregnancy. See Serum progesterone testing to predict ectopic pregnancy in symptomatic first-trimester patients, 36:2 Annals of Emergency Medicine 95-100 (August 2000). In the example research that follows, researchers have tried to use certain threshold levels such as 22 ng/ml or 16.22 ng/ml to measure the risk level of the ectopic pregnancy. Though progesterone cannot definitively diagnose an ectopic pregnancy—it is used in conjunction with hCG levels and ultrasound findings to guide clinical decisions and may guide users to seek more immediate medical attention based on symptom presentation in conjunction with low PdG levels. For that reason, measured quantitative levels of PdG may be compared to various standards when tracking PdG levels.

According to this study, each patient with an ectopic pregnancy had a progesterone level below 22 ng/ml. Of the patients with a progesterone level below 22 ng/ml, 10% had an ectopic pregnancy, but none of the patients with progesterone over 22 had an ectopic pregnancy. Of the patients who did not have an ectopic pregnancy, 73% had a progesterone level below 22 ng/ml. Therefore, a progesterone level above 22 ng/ml indicates that the likelihood of having an ectopic pregnancy according to this study was 2% or less. Further, even if the progesterone level is below 22 ng/ml, the likelihood of not having an ectopic is above 88%.

A further study provides a comparison of progesterone between normal pregnancies and ectopic pregnancies at an average gestational age of 6.6+/−1.5 weeks for normal pregnancies and 6.3+/−1.6 weeks for ectopic pregnancies, shown in the table below. See Serum Progesterone Measurement in Diagnosis of Ectopic Pregnancy, Journal of South Asian Federation of Obstetrics and Gynaecology (2019).

TABLE 3
Comparison of progesterone between normal
pregnancy and ectopic pregnancy

| Unit of | | |
|---|---|---|
| measurement: | Normal | |
| ng/mL | pregnancy | EP |
| Progesterone | 24.6 ± 8.8 | 6.4 ± 3.8 |
| Minimum | 10.32 | 0.58 |
| Maximum | 53.54 | 20.07 |

p value = 0.000

As described, the systems and methods taught herein can be implemented to analyze and compare the quantitative progesterone level trends of each patient compared to: (1) normal progesterone level trends derived from measurements from a large number of women; (ii) from the user's past health data; or (iii) other statistically relevant data sets. As such, known risk factors, such as threshold measurements of hormone levels that have been determined by science research, big data analysis, etc. may be used indicate or assess the risk of ectopic pregnancy, or other health concern, for each user.

For example, FIG. 9 illustrates a chart 208 comparing the “expected” quantitative levels of PdG over the weeks of a normal cycle into the first few weeks of early pregnancy (curve 210) against the PdG levels measured by a woman at risk for ectopic pregnancy (curve 212), the data for either or both curves may have been produced by the test system 100. The chart 208 shown in FIG. 9 may help the user and medical professionals indicate or assess the risk of ectopic pregnancy.

Further, the test system 100 of the present application may be used to screen or assess the risk of ovarian cancer or problems with the adrenal glands. In one study, the progesterone levels were significantly higher in all ovarian tumor groups as compared with the controls. See Elevated Progesterone Levels in Serum and Ovarian Venous Blood in Patients with Ovarian Tumors, 64:8 Acta Obsetricia et Gynecologica Scandinavica, 649-652 (January 1985). There was no difference between malignant and benign tumors. Patients with a tumor volume over 1,000 ml had higher progesterone levels than patients with smaller tumors, in both peripheral and ovarian venous blood.

Similarly, conditions such as ovarian cysts, a disorder of the adrenal glands, or a molar pregnancy, where abnormal cells grow in the placenta, may cause patients to have too much progesterone. See High Progesterone Symptoms and Side Effects by M. Pugle (February 2024). The excess progesterone may additionally cause elevated progesterone levels in the follicular phase of the menstrual cycle. Accordingly, the systems and methods taught herein may provide valuable screening tools for concurrent pathologies associated with endocrinological dysfunction in women who are trying to conceive. As described, the systems and methods taught herein can be used to show, analyze, and compare the quantitative progesterone level trends of each user with respect to the expected, normal, or otherwise relevant progesterone level trends.

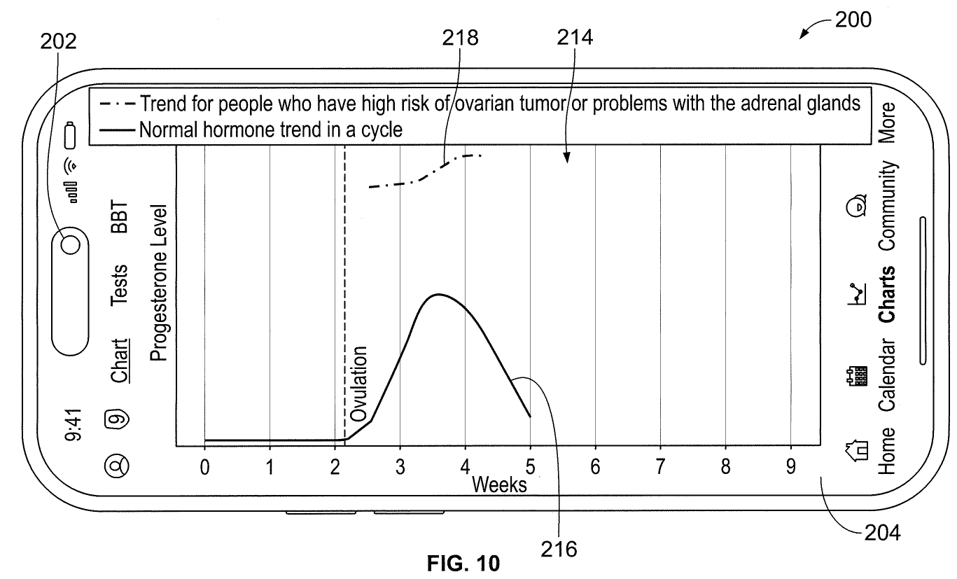

For example, FIG. 10 illustrates a chart 214 comparing the “expected” or normal quantitative levels of PdG over the weeks of a normal cycle that does not lead to pregnancy (curve 216) against the PdG levels measured by a woman at risk for having an ovarian tumor or having problems with her adrenal glands (curve 218), the data for either or both curves may have been produced by the test system 100. The chart 214 shown in FIG. 10 may help the user and medical professionals indicate or assess the risk of having an ovarian tumor or having problems with her adrenal glands.

Still further, the test system 100 of the present application may be used to screen or assess the risk of miscarriage (loss of pregnancy before 20 weeks). One study found that progesterone was a predictor of miscarriage at seven to nine weeks into the gestational period, as the onset of substantial progesterone production is typically the seventh gestational week. As shown in other studies, high progesterone levels on the day of the embryo transfer was an indicator of a higher likelihood of ongoing pregnancy. In another study, serum progesterone concentration increased linearly with gestational age from 5-13 weeks in women with normal pregnancies. In contrast, women with spontaneous miscarriages often showed a marginal, non-significant increase in serum progesterone in this time frame. The results also showed higher progesterone levels at seven to nine gestational weeks than progesterone levels at five to six gestational weeks in normal pregnancy. Similarly, women with miscarriages showed lower progesterone levels than progesterone levels of normal pregnancies at seven to nine gestational weeks. See Prediction of miscarriage in first trimester by serum estradiol, progesterone and β-human chorionic gonadotropin within 9 weeks of gestation by Deng et al., 22:112 BMC Pregnancy and Childbirth (2022). These findings demonstrate that quantitative tracking of PdG could be an early indicator for miscarriage and also may provide insight into the necessity of bioidentical progesterone supplementation. Studies also suggest an important role of progesterone in supporting an early pregnancy. For example, progesterone levels at seven to nine weeks into the gestational period lower than the cutoff value, such as 15.27 ng/ml, may predict miscarriage. Accordingly, the systems and methods taught herein may be used to show, analyze, and compare the quantitative progesterone level trends of each user with respect to the expected, normal, or otherwise relevant progesterone level trends to indicate or assess the risk of miscarriage for each user.

For example, FIG. 11 illustrates a chart 220 comparing the “expected” or normal quantitative levels of PdG over the weeks of a normal cycle that results in pregnancy (curve 222) against the PdG levels measured by a woman at higher risk for having a miscarriage (curve 224), the data for either or both curves may have been produced by the test system 100. The chart 220 shown in FIG. 11 may help the user and medical professionals indicate or assess the risk of miscarriage.

It should be noted that various changes and modifications to the embodiments described herein will be apparent to those skilled in the art. Such changes and modifications may be made without departing from the spirit and scope of the present invention and without diminishing its attendant advantages. For example, various embodiments of the apparatus and methods may be provided based on various combinations of features and functions from the subject matter provided herein.

1. A test system comprising:
a test device including a test area, a results area, and a control area, wherein the test area of the body includes a lateral flow assay that detects the presence of a target analyte in a target substance, wherein, in response to exposure to the target analyte, the results area produces a visually identifiable test-line having a test-line color density and a visually identifiable control-line having a control-line density, wherein the visually identifiable test-line increases in darkness according to an increased concentration level of the target analyte; and
a calibration chart including a plurality of color blocks, wherein each of the color blocks is representative of a test-line color density that corresponds to a quantitative hormone concentration in the target substance.

2. The test system of claim 1, wherein the test area is a non-competitive lateral flow assay for determining the concentration of the target analyte, wherein the target analyte is one or more progesterone metabolite, including a pregnanediol such as PdG, pregnanolone, and pregnanetriol.

3. The test system of claim 1, further comprising:
a user device including a camera, a processor, a display, and memory storing program instructions, wherein, in response to executing the program instructions, the processor:
receives an image of the test area from the camera, the image including the visually identifiable test-line and the visually identifiable control-line;
determines a test-line value defined as a numerical value of a color density of the test-line in the image;
determines a control-line value defined as a numerical value of a color density of the control-line in the image; and
calculates a quantitative hormone concentration in the target substance using the numerical value of the color density of the test-line in the image and the numerical value of the color density of the control-line in the image.

4. The test system of claim 3, wherein calculating a quantitative hormone concentration in the target substance using the numerical value of the color density of the test-line in the image and the numerical value of the color density of the control-line in the image, includes:
calculating a T/C ratio defined as a relative value of the test-line value to the control-line value; and
determining a quantitative hormone level based on the comparison between the T/C ratio and a data structure that maps calibrated T/C ratios to quantitative hormone levels.

5. The test system of claim 4, wherein, in response to executing the program instructions, the processor further presents visualized data through the display indicative of one or more T/C ratio trends or one or more quantitative hormone level trends.

6. The test system of claim 1, further comprising a user device including a processor, a display, and memory storing program instructions, wherein, in response to executing the program instructions, the processor:
receives quantitative hormone levels at one or more specific stages of a menstrual cycle or a pregnancy derived from the test device and calibration chart; and
compares the quantitative hormone levels at one or more specific stages of a menstrual cycle or a pregnancy to one or more normal hormone level trends.

7. The test system of claim 6, wherein the processor further compares the quantitative hormone levels at one or more specific stages of a menstrual cycle or a pregnancy to one or more threshold values associated with risk levels of certain health condition, wherein the one or more threshold values associated with risk levels of one or more health conditions are determined based on scientific research and data analysis in combination with a user's personalized data.

8. The test system of claim 7, wherein the one or more health conditions include: a miscarriage, an ectopic pregnancy, an ovarian tumor, or an adrenal gland problem.

9. The test system of claim 6, wherein the display provides an indication of a potential health condition risk derived from a comparison between the quantitative hormone levels at one or more specific stages of a menstrual cycle or a pregnancy derived from the test device and calibration chart and one or more normal hormonal level trends.

10. A test system comprising:
a test device including a test area, a results area, and a control area, wherein the test area of the body includes a lateral flow assay that detects the presence of a target analyte in a target substance, wherein, in response to exposure to the target analyte, the results area produces a visually identifiable test-line having a test-line color density and a visually identifiable control-line having a control-line density, wherein the visually identifiable test-line increases in darkness according to an increased concentration level of the target analyte; and
a user device including a camera, a processor, a display, and memory storing program instructions, wherein, in response to executing the program instructions, the processor:
receives an image of the test area from the camera, the image including the visually identifiable test-line and the visually identifiable control-line;
determines a test-line value defined as a numerical value of the test-line color density;
determines a control-line value defined as a numerical value of the control-line color density;
calculates a T/C ratio defined as a relative value of the test-line value to the control-line value; and
determines a quantitative hormone level based on the comparison between the T/C ratio and a data structure that maps calibrated T/C ratios to quantitative hormone levels.

11. The test system of claim 10, further wherein the processor presents visualized data through the display indicative of one or more T/C ratio trends or one or more quantitative hormone level trends.

12. The test system of claim 10, wherein the T/C ratios or the quantitative hormone levels at one or more specific stages of a menstrual cycle or a pregnancy are compared to one or more threshold values associated with risk levels of certain health conditions and presented in the visualized data, wherein the one or more threshold values associated with risk levels of one or more health conditions are determined based on scientific research and data analysis in combination with a user's personalized data.

13. The test system of claim 12, wherein the one or more health conditions include: a miscarriage, an ectopic pregnancy, an ovarian tumor, or an adrenal gland problem.

14. The test system of claim 10, wherein the processor presents visualized data through the display including a comparison between the one or more T/C ratio trends or one or more quantitative hormone level trends and one or more normal hormone level trends.

15. The test system of claim 10, wherein the visualized data presented through the display further includes an indication of a potential health condition risk derived from a comparison between the one or more T/C ratio trends or one or more quantitative hormone level trends and one or more normal hormone level trends.
